(19) United States
(12) Patent Application Publication
(10) Pub. No.: US 2025/0205511 A1
(43) Pub. Date: Jun. 26, 2025

(54) SKIN TREATMENT DEVICE

(71) Applicant: c/o iPulse Limited, Office Block A, Swansea (GB)

(72) Inventors: Stuart Terry Jones, Swansea (GB); Paul Edward Davies, Swansea (GB)

(21) Appl. No.: 18/395,229

(22) Filed: Dec. 22, 2023

(30) Foreign Application Priority Data
Jan. 12, 2023 (GB) GB2300486.4

(51) Int. Cl. A61N 5/06

(52) U.S. Cl. CPC A61N 5/0617; A61N 2005/0629; A61N 2005/0644

(57) ABSTRACT

A skin treatment device for delivery of light energy pulses to a subject's skin, including a housing, a light source housed within the housing and a control system for controlling discharge of the light source to deliver the light energy pulses. The housing has an output window for transmission of the light energy pulses emitted by the light source to external of the housing onto the subject's skin. At least one projection projects outwardly from the housing relative to the output window and has a skin contact surface. Each projection is at least partially moveable from a rest position to a deflected position to accommodate the geometry of the subject's skin. The projection further carries a sensor, and the control system is arranged to receive one or more sensor outputs from the sensor and based on the one or more sensor outputs control operation of the skin treatment device.

### CROSS-REFERENCE TO RELATED APPLICATIONS

This application claims the benefit of GB Patent Application No. 2300486.4 filed 12 Jan. 2023, the entire content and substance of which is incorporated herein by reference in its entirety as if fully set forth below.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### SEQUENCE LISTING

Not Applicable

### STATEMENT REGARDING PRIOR DISCLOSURES BY THE INVENTOR OR A JOINT INVENTOR

Not Applicable

### BACKGROUND OF THE DISCLOSURE

### 1. Field of the Invention

The present invention relates to a skin treatment device, preferably a skin treatment device for treating unwanted hair, and preferably comprising an Intense Pulsed Light (IPL) device.

### 2. Description of Related Art

Skin treatment devices are known in the art for treatment of, for example, cosmetic applications such as hair depilation, minimization of skin blemishes or skin rejuvenation, as well as dermatological treatment of skin conditions such as acne or rosacea. The skin is exposed to dosages of radiation from a light source such as a flashlamp or laser where the radiation is targeted to the skin and the energy intensity and pulse duration is controlled. In hair depilation, the radiation source is targeted to cause heating of the hair root causing the hair root to die.

Safety of skin treatment devices is paramount and is particularly important for devices designed for home use. Home use devices require ‘good skin contact’ before allowing the device to emit radiation to the skin. Good contact can be defined as a condition where the light output area is sufficiently covered by skin that any stray optical radiation is below harmful levels. As such, safety features are implemented so that the device will not emit radiation unless the device is in contact with a user's skin to minimize stray optical radiation from the device in operation which may be at unsafe levels for the eyes.

This is typically achieved through the provision of multiple sensors adjacent each side of the output window (for example above, below and to either side of a rectangular output window) in the head of the device where a surface must be detected by each sensor as a requirement for radiation to be emitted. If one sensor does not measure a threshold value, then it is determined by the control system of the device that there is no skin contact and firing is prevented. This is to prevent the device firing when good contact with the skin is not achieved with the associated risk of the emission of potentially harmful levels of stray optical radiation. Stray optical radiation can be defined as light emitted by the device that is intended to be absorbed by the target skin area for treatment purposes, but either misses the intended target, is reflected or remitted from the target.

Various sensors may be utilized such as capacitive contact sensors or proximity sensors. Capacitive contact sensors are often utilized, and FIG. 1 is a schematic illustration showing a skin treatment device 2 comprising a housing 4 shaped to be grasped by a user by a handle 6 having a forward end 8 comprising an output window 10 for emission of light energy pulses onto the skin. Positioned around the output window in the forward end 6 of the housing 4 are multiple sensors 12 capable of detecting skin contact. Assuming a threshold capacitance is measured from the sensors, then the control system in the device does not prevent emission of an energy pulse as it is deemed there is contact with the skin and emission of an energy pulse is safe.

While the safety features of the device limit stray radiation, there is an impact on useability. For body areas with a large flat surface, usability is good as a user can readily position the device such that all of the sensors contact the body thus allowing emission of radiation and ease of treatment. However, on more angular or bony areas of the body it is more difficult to orient the head so that all the sensors are in body contact and emission of radiation is prevented. This causes frustration for the user, and the efficacy of the treatment is reduced as difficult to treat body areas are missed. Overall usability is therefore decreased. This is clearly shown in FIG. 2A where the device can readily treat a relatively flat area such as a user's calf. This compares with FIG. 2B which shows attempted treatment of an arm where the gap 14 between the shin and sensors means the control system prevents emission of a light energy pulse.

Existing solutions have been proposed. For example, adaptors have been utilized that selectively secure to the front end of a device and have a curved leading edge thereby providing better communication with a curved surface of a user. This type of arrangement works for specific body geometries however is limited in suitability meaning multiple different adaptors are required leading to a significant lack of useability. Alternatively, a head portion of a device can in some circumstances be removed and replaced with a head portion having a smaller output transmission window and skin contact surface thereby enabling more complex body geometries to be treated as a result of the smaller contact area. This results in increased treatment time for the areas treated thereby reducing usability.

What is needed, therefore, are systems and methods that address these problems or at least provide a useful alternative.

### BRIEF SUMMARY OF THE INVENTION

According to a first aspect of the present invention there is a skin treatment device for delivery of light energy pulses to a subject's skin, the skin treatment device comprising a housing, a light source housed within the housing for discharging light energy pulses, a control system for controlling discharge of the light source to deliver the light energy pulses, the housing having an output window for transmission of the light energy pulses emitted by the light source to external of the housing onto the subject's skin, the skin treatment device further comprising at least one projection projecting outwardly from the housing relative to the output window and each projection having a skin contact surface, wherein each projection is at least partially moveable from a rest position to a deflected position such that the projection deflects to accommodate the geometry of the subject's skin, the projection further carrying a sensor and where the control system is arranged to receive one or more sensor outputs from the sensor and based on the one or more sensor outputs control operation of the skin treatment device.

It will be appreciated that a skin contact sensor may carry one or more individual sensors.

The skin contact surface preferably faces outwardly from the projection.

The provision of the skin contact portion that carries one or more sensors being at least partially moveable means that the projection(s) can dynamically move to adapt to the geometry of the skin. Undulations in the shape of the body are therefore accommodated and can be treated safely and with ease of operation. This therefore increases usability and at the same time reduces the possibility of stray light escaping. As the projection deflects, the output window can move into a closer proximity to the skin.

A projection beneficially acts as a light guide for guiding the light energy pulses to the skin. Correspondingly, a projection also acts to prevent escape of stray light that has been output from the output window.

It will be appreciated that as the projection moves the carried sensor(s) also moves. The at least one sensor is preferably embedded in the skin contact surface. The sensor is beneficially retained by the projection, and is further beneficially held in a fixed position relative to the skin contact surface. The device preferably comprises at least a first and a second projection, where the first and second projection deflect independently of one another.

It will be appreciated that the at least one sensor is carried by a portion of the projection that deflects.

It will be appreciated that the at least one projection projects forwardly from the housing. This forward projection is in a direction in which light is emitted from the transmission window. The at least one projection acts to guide the light emitted from the output window. The skin contact portion extends forwardly in a direction at least partially in a direction perpendicular to a plane of the transmission window. The at least one projection may extend around the entirety of the output window.

The skin treatment device preferably comprises a plurality of sensors. The plurality of sensors may comprise proximity sensors (such as capacitive sensors) and/or optical proximity sensors depending on the specific functionality required. However, in any event it is beneficial that the sensors can be used to determine proximity of a surface (skin) to the sensor. If an optical proximity sensor is utilized, additional functionality may be provided such as the ability to use the sensor output (reflectance) in determination of the skin tone and therefore control the energy output by the light source dependent upon the skin tone.

The sensor(s) may be a proximity or contact sensor for detecting the presence of skin. An example of a typical sensor utilized in the present device is a capacitive proximity sensor.

The one or more sensors preferably comprises at least first and second sensors carried by the at least one projection on diametrically opposing first and second sides of the output window.

The at least one projection preferably comprises at least a first and second projection independently moveable relative to each other. It will be understood that by providing first and second projections the provision of a third or more projection is not excluded. However, for skin treatment device that is portable and handheld, limitation to two projections only have been found to be beneficial. The first and second projection are each preferably positioned on opposing sides of the output window. The output window preferably comprises a length dimension and a width dimension, where the length dimension is greater than the width dimension, and the length dimension extends between the first and second projections. The first and second projection may comprise first and second jaws. A recessed portion is preferably defined between the first and second portions.

A projection is preferably arranged to be received into a corresponding opening in the housing, where the projection in the rest position extends outwardly from the opening, and in the deflected position the projection is at least partially withdrawn into the opening.

The at least one projection is preferably rigid. This means that the at least one projection itself is not deformable. The at least one projection is beneficially biased to the rest position by a biasing arrangement. The biasing arrangement preferably comprises a spring.

The at least one projection is preferably pivotally mounted to the housing. The skin contact surface preferably comprises a leading edge and a trailing edge, where the leading edge projects further outwardly from the housing in the rest position. The trailing edge is beneficially closer to the output window than the leading edge. The leading edge (and/or trailing edge) may be generally parallel to an edge of the output window.

The housing has a forward contact surface for contacting a user's skin, where the output window is defined in the forward contact surface. The forward contact surface is at least partially convex. The output window preferably has a solid window therein for allowing emission of light from the housing, and the solid window may also be contoured to match the geometry of the forward contact surface.

The at least one projection may be elastically deformable and conform to the geometry of a user's skin. The at least one projection may comprise an elastomeric material such as rubber. The at least one projection may extend around the entire output window. The at least one projection may comprise a single integrated projection.

Controlling operation of the device may comprise one or both of determining whether or not the flashlamp can emit a light energy pulse and determining properties of that pulse (e. g. fluence).

The at least one projection is preferably non-detachable from the housing under normal operational use.

The device is preferably an Intense Pulsed Light (IPL) device.

According to a second aspect of the present invention there is a skin treatment device for delivery of light energy pulses to a subject's skin, the skin treatment device comprising a housing having an output window, a light source housed within the housing for discharging light energy pulses transmitted through the output window onto the subject's skin, a control system for controlling discharge of the light source to deliver the light energy pulses, and one or more projections projecting outwardly from the housing relative to the output window, at least one projection having a skin contact surface and carrying a sensor, wherein at least one projection is at least partially moveable from a rest position to a deflected position such that the respective at least one projection deflects to accommodate the geometry of the subject's skin, and wherein the control system is arranged to receive one or more sensor outputs from the sensor of at least one projection, and based on one or more of the sensor outputs, control operation of the skin treatment device.

In any embodiment of the present invention, the skin contact surface faces outwardly from at least one projection.

In any embodiment of the present invention, the sensor of at least one projection is retained by the respective projection and is further held in a fixed position relative to the respective skin contact surface.

In any embodiment of the present invention, a first projection of the one or more projections and a second projection of the one or more projections deflect independently of one another.

In any embodiment of the present invention, at least one projection extends around the entirety of the output window.

In any embodiment of the present invention, the sensor of a first projection of the one or more projections and the sensor of a second projection of the one or more projections are carried on diametrically opposing first and second sides of the output window.

In any embodiment of the present invention, the output window comprises a length dimension and a width dimension, wherein the length dimension is greater than the width dimension, and wherein the length dimension extends between two projections of the one or more projections.

In any embodiment of the present invention, at least one projection is arranged to be received into a corresponding opening in the housing, wherein at least one received projection in the rest position extends outwardly from the opening, and wherein at least one received projection in the deflected position is at least partially withdrawn into the opening.

In any embodiment of the present invention, at least one projection is rigid.

In any embodiment of the present invention, at least one projection is beneficially biased to the rest position by a biasing arrangement.

In any embodiment of the present invention, the housing has a forward contact surface for contacting a user's skin, wherein the output window is defined in the forward contact surface, and wherein the forward contact surface is at least partially convex.

In any embodiment of the present invention, at least one projection comprises a unitary projection.

In any embodiment of the present invention, at least one projection is pivotally mounted to the housing.

In any embodiment of the present invention, at least one projection is elastically deformable and arranged to conform to the geometry of a user's skin.

In any embodiment of the present invention, the skin contact surface of at least one projection comprises a leading edge and a trailing edge, wherein the leading edge projects further outwardly from the housing in the rest position.

In any embodiment of the present invention, at least one projection comprises an elastomeric material.

According to a third aspect of the present invention there is a skin treatment device for delivery of light energy pulses to a subject's skin, the skin treatment device comprising a housing having an output window, a light source housed within the housing for discharging light energy pulses transmitted through the output window onto the subject's skin, a control system for controlling discharge of the light source to deliver the light energy pulses, and one or more projections projecting outwardly from the housing relative to the output window, each projection having a skin contact surface and carrying a sensor, wherein each projection is at least partially moveable from a rest position to a deflected position such that each projection deflects to accommodate the geometry of the subject's skin, and wherein the control system is arranged to receive one or more sensor outputs from the sensor of at least one projection, and based on one or more of the sensor outputs, control operation of the skin treatment device.

In any embodiment of the third aspect of the present invention, at least one of the control system is arranged to receive one or more sensor outputs from the sensor of each projection, and based on the one or more of the sensor outputs, control operation of the skin treatment device, the skin contact surface faces outwardly from each projection, the sensor of each projection is retained by the projection and is further held in a fixed position relative to the skin contact surface, or wherein a first projection of the one or more projections and a second projection of the one or more projections deflect independently of one another.

In any embodiment of the third aspect of the present invention, at least one of the sensor of a first projection of the one or more projections and the sensor of a second projection of the one or more projections are carried on diametrically opposing first and second sides of the output window, the output window comprises a length dimension and a width dimension, the length dimension greater than the width dimension, and the length dimension extending between two projections of the one or more projections, each projection is arranged to be received into a corresponding opening in the housing, each projection in the rest position extending outwardly from the opening, and each projection in the deflected position being at least partially withdrawn into the opening, each projection is rigid, each projection is beneficially biased to the rest position by a biasing arrangement, each projection comprises a unitary projection, each projection is pivotally mounted to the housing, or each projection is elastically deformable and arranged to conform to the geometry of a user's skin.

In any embodiment of the third aspect of the present invention, at least one of the housing has a forward contact surface for contacting a user's skin, the output window defined in the forward contact surface, and the forward contact surface being at least partially convex, each projection is pivotally mounted to the housing, the skin contact surface of each pivotally mounted projection comprising a leading edge and a trailing edge, the leading edge projecting further outwardly from the housing in the rest position, or each projection is elastically deformable and arranged to conform to the geometry of a user's skin, each elastically deformable projection comprising an elastomeric material.

These and other aspects of the present invention are described in the Detailed Description below and the accompanying figures. Other aspects and features of embodiments of the present invention will become apparent to those of ordinary skill in the art upon reviewing the following description of specific, exemplary embodiments of the present invention in concert with the figures. While features of the present invention may be discussed relative to certain embodiments and figures, all embodiments of the present invention can include one or more of the features discussed herein. Further, while one or more embodiments may be discussed as having certain advantageous features, one or more of such features may also be used with the various embodiments of the invention discussed herein. In similar fashion, while exemplary embodiments may be discussed below as device, system, or method embodiments, it is to be understood that such exemplary embodiments can be implemented in various devices, systems, and methods of the present invention.

### BRIEF DESCRIPTION OF THE DRAWINGS

The accompanying drawings, which are incorporated in and constitute a part of this specification, illustrate multiple embodiments of the presently disclosed subject matter and serve to explain the principles of the presently disclosed subject matter. The drawings are not intended to limit the scope of the presently disclosed subject matter in any manner.

FIG. 1 presents a skin treatment device describing the general functionality of a known skin treatment device.

FIGS. 2A-2B demonstrate the ease of treatment of a relatively flat skin area compared to curved area of the skin for a prior art device.

Referring to FIGS. 3A-3B there is perspective view of a device and a forward end of a device according to an illustrative embodiment of the present invention.

FIGS. 4A-4B show the mechanism by which the projections can deflect relative to the body of the device, where the body of the device has been removed for clarity purposes.

FIGS. 5A-5C show an embodiment of the present invention is presented in side view, perspective view and cross sectional view respectively. Each view shows the device in three different operational states depending on the curvature of the body.

FIG. 6 is a further illustrative embodiment of the present invention.

FIGS. 7A-7B is a schematic representation of an illustrative embodiment of the present invention in a rest and deflected configuration respectively.

FIGS. 8A-8B is a schematic representation of an illustrative embodiment of the present invention.

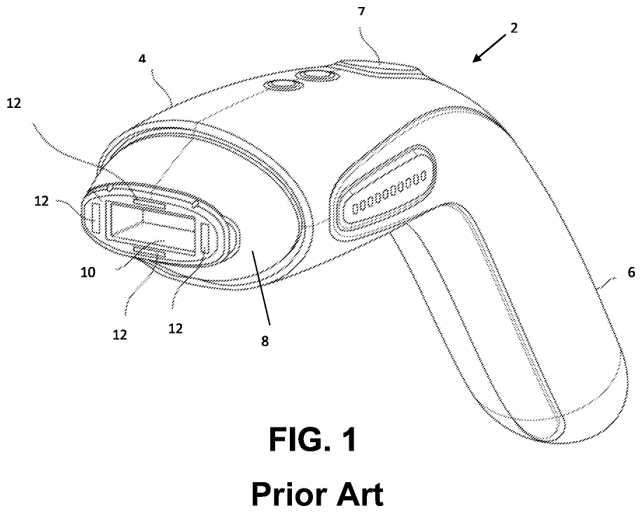

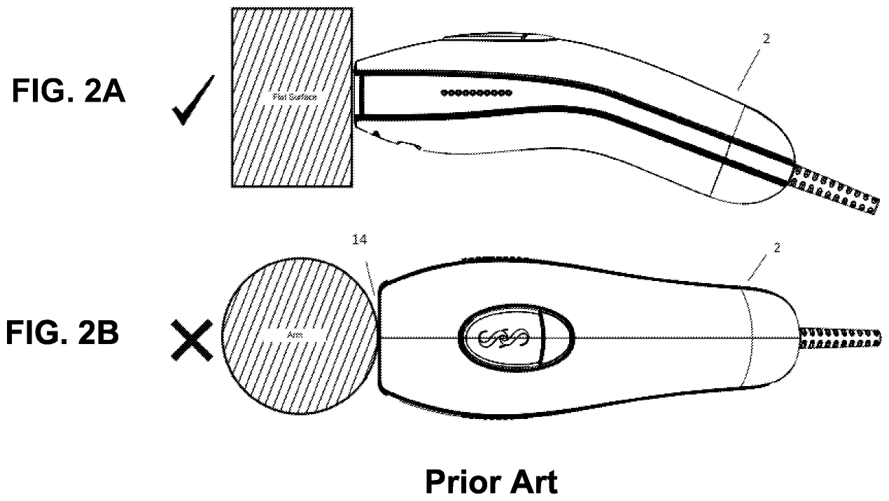

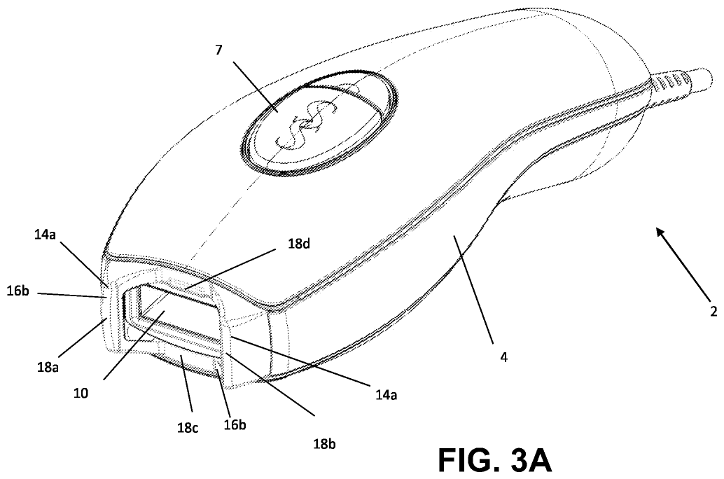

### DETAILED DESCRIPTION OF THE INVENTION

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

It should also be noted that, as used in the specification and the appended claims, the singular forms “a,” “an,” and “the” include plural references unless the context clearly dictates otherwise. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is also to be understood that the mention of one or more method steps does not preclude the presence of additional method steps or intervening method steps between those steps expressly identified. Moreover, although the term “step” may be used herein to connote different aspects of methods employed, the term should not be interpreted as implying any particular order among or between various steps herein disclosed unless and except when the order of individual steps is explicitly required.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter. Additionally, the components described herein may apply to any other component within the disclosure. Merely discussing a feature or component in relation to one embodiment does not preclude the feature or component from being used or associated with another embodiment.

To facilitate an understanding of the principles and features of the disclosure, various illustrative embodiments are explained below. Referring to FIGS. 3A-3B schematically presented is the entire device and forward end respectively of a skin treatment device that may be used for treating skin disorders and conditions, and even more beneficially is suitable for cosmetic purposes such as hair depilation. The device 2 comprises a housing 4 and a light source (not shown) accommodated by the housing 4 such as a discharge lamp or flashlamp. The flashlamp is arranged to generate high intensity pulses of optical radiation. The housing 4 comprises a handle 6 meaning that the housing 4 can be manipulated to be positioned appropriately on a user and is particularly suitable for the home market as the device is handheld, relatively small and portable. A light output window 10 or transmission window 10 allows the passage of high intensity pulses of optical radiation therethrough, where the window 10 typically measuring 30 mm in width and 10 mm in height. The cross-sectional area of the light output aperture/transmission/output window 10 is effectively the treatment area. The light emitting element is housed by the housing 4 together with the control system that controls discharge of the light emitting element.

The head portion of the device 2 in one illustrative embodiment comprises a first projection 14a and second projection 14b arranged on opposing sides of the output window 10, with each protrusion providing a skin contact surface 16a, 16b. In use the skin contact surface bears against the user's skin. The first and second projections 14a, 14b define opposing jaws between which the body portion of the user is received. Within the projections 14a, 14b are first and second sensors 18a, 18b which for providing associated sensing zones. The sensors are embedded into the projections and form part of the skin contact surface of the projections. Additionally, third and fourth sensors 18c, 18d are provided on opposing sides of the output window 10 (which surface also bears against a user's skin in operation) within the forward end of the housing 4 itself.

The sensors may take different forms dependent upon the device in which it is utilized. For example, the sensors may simply comprise multiple proximity sensors in the form of capitative proximity/contact sensors each having a sensing zone where the control system requires a predetermined capacitance to be measured from each sensing zone which is indicative of contact with a user's skin. Accordingly, the sensors may be embedded into the surface, project from the surface or sit behind the surface. In the illustrative embodiment these sensors are 18a-18c and are embedded. Assuming a threshold value is measured, then the control system enables firing of the flashlamp to emit a light energy pulse. However, one or more alternative or additional skin parameters may be sensed, and in the illustrative embodiment this is sensor 18d. For example, this sensor 18d may comprise an optical reflectance sensor often referred to as a skin tone sensor or sometimes again a proximity sensor and can be used in the alternative to or in tandem with one or more other sensor types such as capacitive sensors. In the embodiment presented, there are three capacitive proximity sensors and one optical proximity sensor (or ‘skin tone sensor’ 18d). A skin tone sensor includes a transmitter arranged to transmit sensing radiation through the sensor window onto the skin to be treated. The sensor 18d further includes a receiver such as a photodiode arranged to receive radiation reflected from a skin surface. Intensity of the received radiation is found to be representative of the tone of the skin, for example a light skin tone will reflect more than a dark skin tone. The intensity of the received radiation can be processed by the control system using a processor provided thereby and compares the intensity with a calibrated set of intensity measurements to determine a sensed skin tone, which is then stored in a memory of the control system. The treatment light pulse energy then outputted to the skin can be controlled and is thus dependent on the sensed skin tone thus ensuring optimized treatment for the specific skin tone to be treated.

The projections 14a, 14b each deflect between the rest and deflected position independently of one another. The projections 14a, 14b are each positioned on opposing sides of the output window 10. The output window 10 comprises a length dimension and a width dimension where the length dimension is greater than the width dimension. The length dimension is defined between opposing spaced apart ends and the width dimension is also defined between opposing spaced apart ends. The first and second projections 14a, 14b are positioned so that they are outwardly of the opposing ends. The first and second projection 14a, 14b effectively form first and second jaws, and a recess is defined between the first and second portions. Each of the projections 14a, 14b extend outwardly from the housing 4 and provide a skin contact surface 20a, 20b at a furthermost projection. The shape of the projections tapers inwardly towards the housing 4 and toward the other projection lengthwise parallel to the opposing ends of the width dimensions. This tapered portion 22a, 22b also provides a skin contact surface, and by providing such a shape the possibility of light escaping during operation is minimized. The skin contact surface 20a, 20b may have a tactile surface finish to enable sliding over the skin. Such a surface finish may comprise silicon for example.

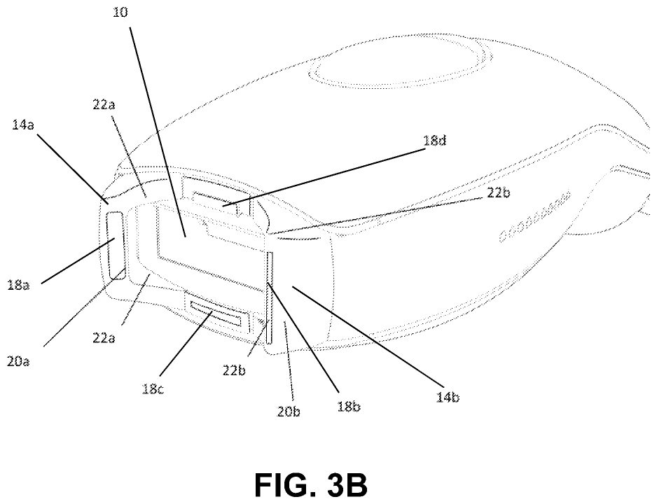

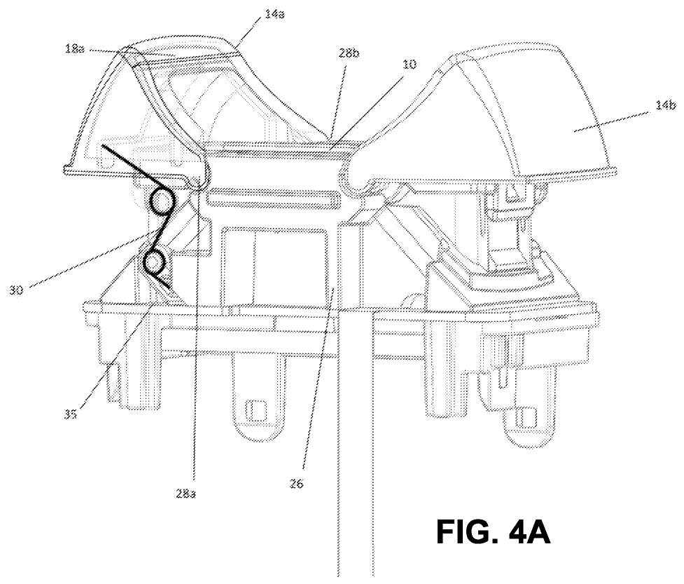

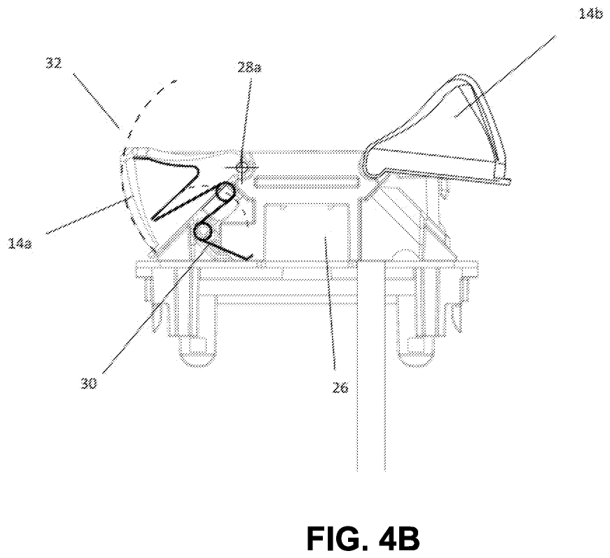

Referring now to FIGS. 4A-4B, the mechanism by which the projections can deflect relative to the body of the device according to an illustrative embodiment is demonstrated, where FIG. 4A shows the projections in the rest or fully extended configuration, and FIG. 4B demonstrates one of the projections in the fully depressed configuration. It will be appreciated that most of the device has been removed for clarity purposes. FIG. 4A shows both projections 14a, 14b in the rest/extended configuration, and projection 14a is shown in a see-through manner so that all components can be readily identified. It will be understood that both projections 14a, 14b are received into corresponding recesses in the housing 4 in the deflected position, which recesses are not shown in FIG. 4.

The projection 14a is pivotally mounted to frame 26 at first and second pivot points 28a, 28b located at opposing sides of the width dimension of the output window 10. A biasing element 30 in the form of a double coil spring is provided for biasing the projection 14a toward the rest/extended configuration as shown in FIG. 4A. Referring to FIG. 4B, the spring is biased toward the contracted configuration by action of a force on the contact surface 20a and the projection 14a has therefore arced as shown by dashed line 32 into a fully deflected configuration. Upon removal of the applied force, the spring 30 returns the projection 14a to the extended configuration. User body portions that do not bias the projections to the fully deflected state are accommodated for by the spring 30 which provides a proportional reaction force to the associated projection ensuring the skin contact surface 20a, 20b aligns as closely as possible to the user body portion.

Further, the embodiment presented in FIG. 4 has the optional benefit of allowing transmission of a sensor output through the described functional components. For example, in one embodiment the output of the sensor 18a can be transmitted through the spring to a receiving circuit 35 that interfaces with the control system.

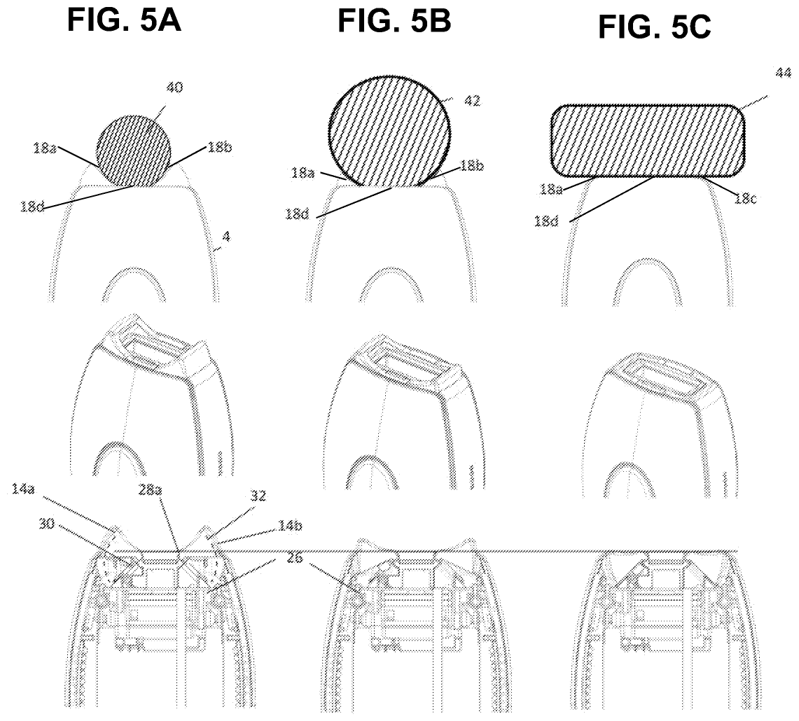

Referring now to FIGS. 5A-5C, an embodiment of the present invention is presented in three operational states respectively, where a side view, perspective view and cross-sectional view is shown. In FIG. 5A, a highly curved surface is contacted that may for example by a user's shin bone 40. The approximate diameter of the body portion may be around 40 mm. When communicating with such a body part, the projections 14a, 14b are fully extended and are in a rest configuration and where operation is the device is enabled as all sensors 18a-d are in close communication with the body portion. Furthermore, stray light emitted from the output window will be minimized. Referring to FIG. 5B, the body part 42 has a medium curvature of approximately 100 mm diameter and may be a different part of a user's leg. The projections 14a, 14b are partially deflected again meaning that good body contact is ensured, and the device can emit an energy pulse to the skin. Referring to FIG. 5C, the body portion 44 is completely flat and as such the projections 14a, 14b are fully deflected and a generally planar contact surface is presented onto the skin of the user.

It will be appreciated that in alternative embodiments an alternative number of projections may be disposed around the output window. As an example, additional projections may be disposed above and below the output window transversely (i. e. across the width).

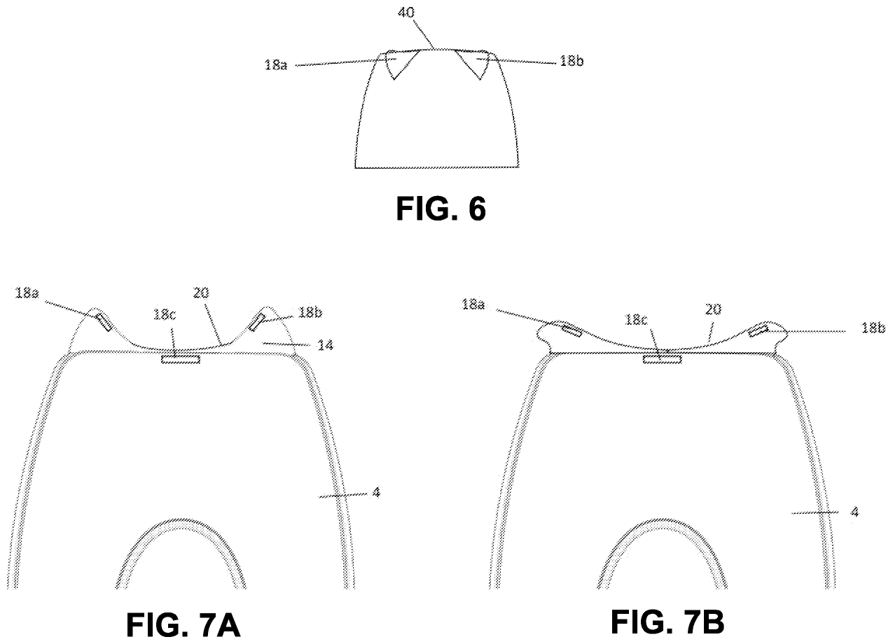

It will be appreciated that there are other body portions with geometries that lie outside of the extremes presented in FIGS. 5A-5C. In alterative embodiments the extremes of movement would be re-defined to accommodate such body portions. For example, referring to FIG. 6, in an illustrative embodiment the user contact surface of a forward end 40 of the housing may be curved, with this curvature being convex in nature to accommodate convex user body portions, such as the inside of the knee. The projections 14a, 14b are shown in the deflected configuration.

In an alternative embodiment of the skin treatment device the one or more projection has a flexible instead of rigid geometry. For example, referring to FIG. 7, a single flexible projection is provided and carries the sensors 18a-d. In this embodiment the entirety of the projection 14 deflects from the rest configuration shown in FIG. 7A to the deflected configuration as shown in FIG. 7B. User body portions that do not conform to the “neutral” or “maximum” states of engagement are accommodated for by flexure of the projection 14, ensuring the skin contact surface 20 conforms to the user body portion. The flexible protrusion in a such an embodiment could be manufactured from an elastomeric material and mechanically or chemically interlocked with the housing 4.

In another embodiment, there are a plurality of flexible projections like the embodiment as presented in FIGS. 3-6 utilizing a pair of opposing flexible projections 14a, 14b on opposing sides of the output window 10 capable of independently deflecting to conform to the body geometry of a user.

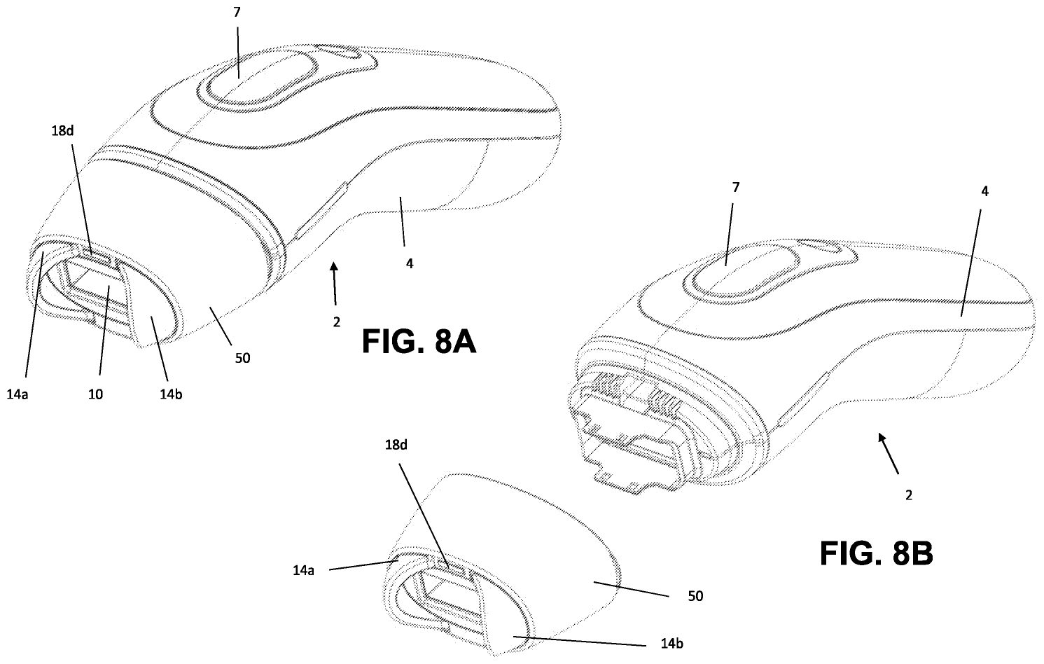

Referring to FIGS. 8A-8B, another embodiment is presented whereby projections 14a and 14b are carried by a head 50 configured to releasably couple to the housing 4. FIG. 8A shows the head 50 coupled to the housing 4, and FIG. 8B shows the head decoupled from the housing 4. In a coupled configuration the device is operable, and in a decoupled configuration the device is configured to be inoperable. There are various benefits associated with such an arrangement. For example, multiple heads may be provided whereby different heads have different size output windows 10 enabling use of the appropriate head for treatment of a particular body area, where a smaller output window 10 is suitable for more awkward body areas such as upper lip. A replacement head may or may not require the provision of the projections 14a, 14b. Alternatively a replacement head could have a different version of the projections 14a, 14b such as those described with respect to FIG. 7.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. Instead, it is intended that the invention is defined by the claims appended hereto.

What is claimed is:

1. A skin treatment device comprising:
a housing having an output window;
a light source housed within the housing and configured to discharge light energy pulses transmittable through the output window and onto a subject's skin;
a control system configured to control the light source to deliver the light energy pulses; and
one or more projections projecting outwardly from the housing relative to the output window, at least one projection having a skin contact surface and carrying a sensor;
wherein at least one projection is at least partially moveable from a rest position to a deflected position such that the respective at least one projection deflects to accommodate the geometry of the subject's skin; and
wherein the control system is further configured to receive one or more sensor outputs from the sensor of at least one projection, and based on one or more of the sensor outputs, control operation of the skin treatment device.

2. The skin treatment device according to claim 1, wherein the skin contact surface faces outwardly from at least one projection.

3. The skin treatment device according to claim 1, wherein the sensor of at least one projection is retained by the respective projection and is further held in a fixed position relative to the respective skin contact surface.

4. The skin treatment device according to claim 1, wherein a first projection of the one or more projections and a second projection of the one or more projections deflect independently of one another.

5. The skin treatment device according to claim 1, wherein at least one projection extends around the entirety of the output window.

6. The skin treatment device according to claim 1, wherein the sensor of a first projection of the one or more projections and the sensor of a second projection of the one or more projections are carried on diametrically opposing first and second sides of the output window.

7. The skin treatment device according to claim 1, wherein the output window comprises a length dimension and a width dimension;
wherein the length dimension is greater than the width dimension; and
wherein the length dimension extends between two projections of the one or more projections.

8. The skin treatment device according to claim 1, wherein at least one projection is arranged to be received into a corresponding opening in the housing;
wherein at least one received projection in the rest position extends outwardly from the opening; and
wherein at least one received projection in the deflected position is at least partially withdrawn into the opening.

9. The skin treatment device according to claim 1, wherein at least one projection is rigid.

10. The skin treatment device according to claim 1, wherein at least one projection is beneficially biased to the rest position by a biasing arrangement.

11. The skin treatment device according to claim 1, wherein the housing has a forward contact surface for contacting the subject's skin;
wherein the output window is defined in the forward contact surface; and
wherein the forward contact surface is at least partially convex.

12. The skin treatment device according to claim 1, wherein at least one projection comprises a unitary projection.

13. The skin treatment device according to claim 1, wherein at least one projection is pivotally mounted to the housing.

14. The skin treatment device according to claim 1, wherein at least one projection is elastically deformable and arranged to conform to the geometry of the subject's skin.

15. The skin treatment device according to claim 13, wherein the skin contact surface of the at least one projection comprises a leading edge and a trailing edge;
wherein the leading edge projects further outwardly from the housing in the rest position.

16. The skin treatment device according to claim 14, wherein the at least one projection comprises an elastomeric material.

17. A skin treatment device for delivery of light energy pulses to a subject's skin, the skin treatment device comprising:
a housing having an output window;
a light source housed within the housing for discharging light energy pulses transmitted through the output window onto the subject's skin;
a control system for controlling discharge of the light source to deliver the light energy pulses; and
one or more projections projecting outwardly from the housing relative to the output window, each projection having a skin contact surface and carrying a sensor;
wherein each projection is at least partially moveable from a rest position to a deflected position such that each projection deflects to accommodate the geometry of the subject's skin; and
wherein the control system is arranged to receive one or more sensor outputs from the sensor of at least one projection, and based on one or more of the sensor outputs, control operation of the skin treatment device.

18. The skin treatment device according to claim 17, wherein at least one of:
the control system is arranged to receive one or more sensor outputs from the sensor of each projection, and based on the one or more of the sensor outputs, control operation of the skin treatment device;
the skin contact surface faces outwardly from each projection;
the sensor of each projection is retained by the projection and is further held in a fixed position relative to the skin contact surface; or
wherein a first projection of the one or more projections and a second projection of the one or more projections deflect independently of one another.

19. The skin treatment device according to claim 17, wherein at least one of:
the sensor of a first projection of the one or more projections and the sensor of a second projection of the one or more projections are carried on diametrically opposing first and second sides of the output window;
the output window comprises a length dimension and a width dimension, the length dimension greater than the width dimension, and the length dimension extending between two projections of the one or more projections;
each projection is arranged to be received into a corresponding opening in the housing, each projection in the rest position extending outwardly from the opening, and each projection in the deflected position being at least partially withdrawn into the opening;
each projection is rigid;
each projection is beneficially biased to the rest position by a biasing arrangement;
each projection comprises a unitary projection;
each projection is pivotally mounted to the housing; or
each projection is elastically deformable and arranged to conform to the geometry of the subject's skin.

20. The skin treatment device according to claim 17, wherein at least one of:
the housing has a forward contact surface for contacting the subject's skin, the output window defined in the forward contact surface, and the forward contact surface being at least partially convex;
each projection is pivotally mounted to the housing, the skin contact surface of each pivotally mounted projection comprising a leading edge and a trailing edge, the leading edge projecting further outwardly from the housing in the rest position; or
each projection is elastically deformable and arranged to conform to the geometry of the subject's skin, each elastically deformable projection comprising an elastomeric material.
